(19) United States
(12) Patent Application Publication
(10) Pub. No.: US 2015/0164418 A1
(43) Pub. Date: Jun. 18, 2015

(54) COGNITIVE PERFORMANCE ASSESSMENT TEST

(71) Applicant: Mayo Foundation for Medical Education and Research, Rochester, MN (US)

(72) Inventors: Bruce D. Johnson, Rochester, MN (US); Robert J. Wentz, Rochester, MN (US); Amine N. Issa, Rochester, MN (US)

(73) Assignee: Mayo Foundation for Medical Education and Research, Rochester, MN (US)

(21) Appl. No.: 14/572,280

(22) Filed: Dec. 16, 2014

Related U.S. Application Data
(60) Provisional application No. 61/916,940, filed on Dec. 17, 2013.

(51) Int. Cl. A61B 5/00; G09B 5/02

(52) U.S. Cl. CPC A61B 5/4884; G09B 5/02

(57) ABSTRACT

This document provides systems and methods for testing and assessing cognitive performance of a user over a period of time in order to test environmental effects and other factors on the performance of the user. For example, cognitive performance over a period of time can be assessed for users experiencing stress conditions including hypoxia (oxygen deprivation), hypothermia, sleep deprivation, fatigue or other factors that may cause degradation in cognitive performance. Furthermore, the systems and methods described herein can be used to identify when hypoxia has manifested in the cerebral circulation of an individual rather than merely in the extremities of an individual.

### CROSS-REFERENCE TO RELATED APPLICATIONS

This application claims the benefit of U.S. Provisional Application Ser. No. 61/916,940, filed Dec. 17, 2013. The disclosure of the prior application is considered part of (and is incorporated by reference in) the disclosure of this application.

### BACKGROUND

1. Technical Field

This document relates to systems and methods for assessing cognitive performance of a user at various points in time. For example, cognitive performance for a user can be tracked along with physiologic biometrics during the course of a specific stressor.

2. Background Information

Persons who are required to engage in complicated tasks under high pressure situations may be exposed to various stresses, impediments, distractions, or outside forces that can affect their cognitive performance. It is therefore desirable to test cognitive performance of a user under various training and testing scenarios in order to assess the user's ability to perform tasks under various conditions.

### SUMMARY

This document provides systems and methods for testing and assessing cognitive performance of a user over a period of time in order to test environmental effects and other factors on the performance of the user. For example, cognitive performance over a period of time can be assessed for users experiencing stress conditions including hypoxia (oxygen deprivation), hypothermia, fatigue, sleep deprivation, concussion or other factors that may cause degradation in cognitive performance. Furthermore, the systems and methods described herein can be used to identify when hypoxia has manifested in the cerebral circulation of an individual before they reach a critical phase of useful consciousness.

Various advantages of the cognitive assessment test described herein include the following. The game will be entertaining and provide an engaging environment in which a user will be tested seamlessly without feeling the pressure of being tested. Users are more likely to perform at their maximum if they are fully engaged in the task at hand. The game can be modifiable to fit the needs of the experiment and the subject population. The game can be used as a competitive arena or as an enjoyable relaxed test depending on the need due to adjustable difficulty tuning. The game can use a learning algorithm to accurately identify a player's skill level, and can be used in a competitive arena to improve performance and motivate users to perform well. Psychological metrics to be assessed can be modified with changes of in-game obstacles to meet the demands of the experiment. The game can display cognitive metrics of performance in real-time. These metrics can include but are not limited to: accuracy, response time, speed, and overall performance. A replay of the game can also be viewable afterwards and the performance can be analyzed in detail along with numerical data. The game can test hand-eye coordination, speed, response time, spatial awareness, situational awareness, valuation, decision making, color detection proficiency, and potentially auditory awareness. The game can offer an instantaneous cognitive performance metric in non-standard environments. This allows for real-time monitoring of cognitive performance when situations (psychological or physiologic) are constantly changing. The game can be on a modifiable platform so that it will continue to evolve and meet the needs of testers.

### DESCRIPTION OF THE DRAWINGS

FIG. 1 is a display screen of a cognitive performance assessment game.

FIG. 2 is a display screen of the cognitive performance assessment game of FIG. 1 that includes a display of cognitive performance metrics.

FIG. 3 is a flow chart of an example process for using a cognitive performance assessment game to assess cognitive performance.

### DETAILED DESCRIPTION

This document provides systems and methods for testing and assessing cognitive performance of a user over a period of time in order to test environmental effects and other factors such as fatigue and mental condition on the performance of the user. For example, cognitive performance over a period of time can be assessed for users experiencing stress conditions including hypoxia (oxygen deprivation), hypothermia, sleep deprivation, mild to extreme fatigue, concussion, or other factors that may cause degradation in cognitive performance. The systems and methods described can also test the effects of various mental conditions or states on cognitive performance, including mild to extreme mental stress or pressure. Furthermore, the systems and methods described herein can be used to identify when hypoxia has manifested in the cerebral circulation of an individual before they reach a critical phase of useful consciousness.

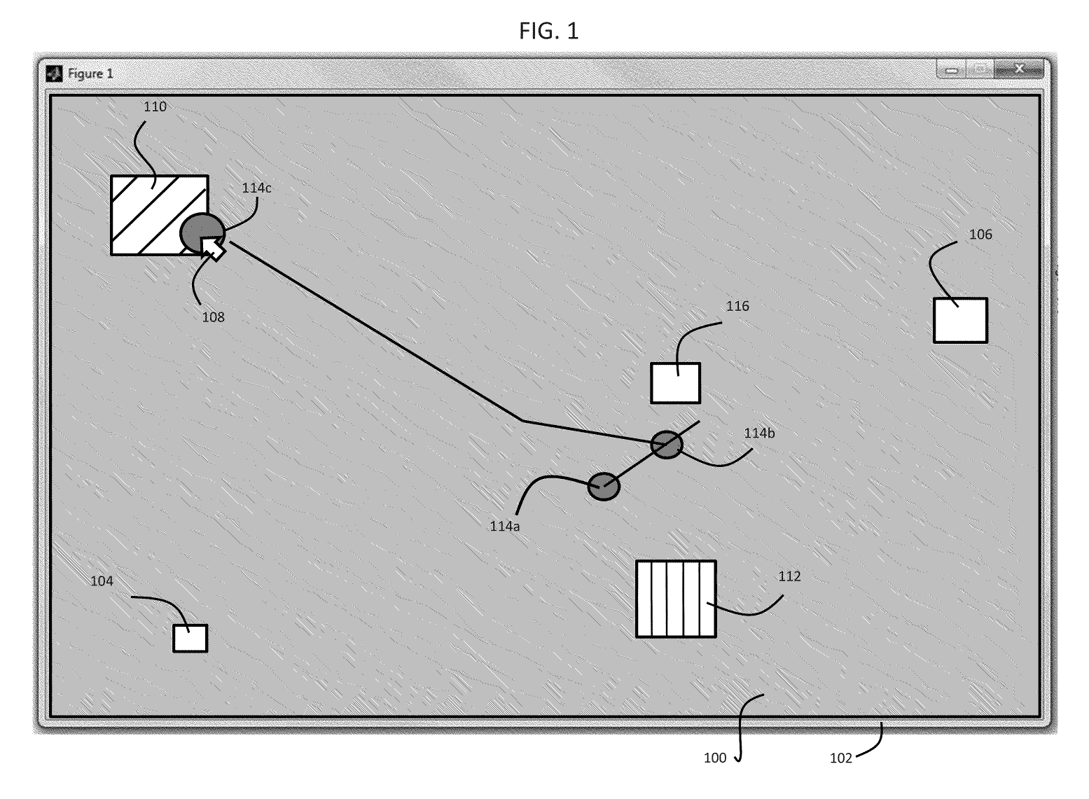

FIG. 1 shows a display screen 100 of a cognitive performance assessment game 102. The cognitive performance assessment game 102 can gage performance with real-time feedback in a variety of environments. The cognitive performance assessment game 102 can engage the user and give insight to several cognitive metrics that are integrated with physiologic metrics under varying conditions of stress, both mental and physical. The cognitive performance assessment game 102 can be used to assess the totality of human performance of an individual by combining physiologic and cognitive metrics to assess performance.

The cognitive performance assessment game 102 of FIG. 1 is played by a user in order to assess the cognitive performance of the user throughout the course of the game. In various embodiments, the user can interact with the cognitive performance assessment game 102 using one or more of a keyboard, a mouse, a controller (e.g. a joystick), voice command software, a motion detection system, eye movement/gaze tracking, a touch screen, or other tactile or aural inputs. For example, a system running the cognitive performance assessment game 102 can monitor eye movement of the user to detect direct gaze selection of various displayed objects and/or icons by the user. In the example shown, the game includes displaying a steady stream of boxes on the display screen 100. For example boxes 104 and 106 are displayed on the display screen 100. In some implementations, the boxes can change in size. For example, a box can start out at a smaller size, such as box 104, and grow to a larger size, such as box 106. In some implementations, boxes can move for a few seconds before disappearing.

The cognitive performance assessment game 102 can give a user a predetermined amount of time to “click” as many boxes as possible. For example, the user can select or “click” on displayed boxes using a cursor 108. As another example, eye tracking functionality can be used to track eye motions of the user and the user can select displayed boxes by looking at the displayed boxes. In some implementations, the predetermined amount of time is kept short, such as for example, one or two minutes. When the user clicks a box, it disappears and the user is awarded points. In some implementations, if a box is not clicked by the user within a specified time period (e.g., 1-2 seconds) the box disappears and the user loses points. If the user attempts to click a box and misses, the user loses points.

The cognitive performance assessment game 102 can also display boxes of varying colors. For example, the boxes 104 and 106 can be presented as grey boxes while a box 110 can be a yellow box. The cognitive performance assessment game 102 can be configured to display grey boxes more frequently than yellow boxes. For example, several grey boxes can be displayed per second, while yellow boxes are displayed at predetermined or random intervals. For example, a yellow box can be displayed every 8 seconds. Displaying boxes of different colors can help the user retain focus and diminish effects of decision fatigue.

In some implementations, if the user successfully clicks the yellow box 110, the user can be awarded bonus points (i.e., additional points on top of a normal allotment of points given for successful selection of a box of a different color, such as a grey box). In some implementations, a successful click on the yellow box 110 can cause a blue box 112 to be displayed. Clicking on the blue box 112 can lead to an even higher amount of bonus points being added to the user's score. In some implementations, if the user does not click the yellow box 110 before a predetermined time period has expired (e.g., 0.5 seconds), the yellow box 110 can change colors (e.g., from yellow to red) and the user loses points when clicking on the now red box 110. In some implementations, rather than displaying different colored boxes, as described in the above example, the cognitive performance assessment game 102 can display graphic icons of different shapes (e.g., squares, triangles, circles, and stars) or graphic icons having different visual patterns. In some implementations, if a displayed graphic icon has not been selected within a predetermined time duration, rather than changing the color of the graphic icon, the cognitive performance assessment game 102 can change the shape of the graphic icon, a visual pattern of the graphic icon, or another display aspect of the visual icon.

In some implementations, a red dot is displayed on the display screen 100 whenever the user selects a location on the display screen 100. For example, the dots 114a-c can be displayed to indicate that the user has clicked on the three indicated locations on the display screen 100. This can help the user to identify how far off the user was when the user misses a box and help the user to improve future performance. For example, the dot 114b indicates a location clicked on by the user using the cursor 108 while attempting to click on a box 116. By displaying the dot 114b, the cognitive performance assessment game 102 indicates to the user how far off the user was when the user missed the box 116. Additionally, the dots 114a-c can add an additional visual stimulus to the game that must be ignored by the user when cognitive performance is being tested.

In some implementations, the cognitive performance assessment game 102 can include audio stimuli in addition to visual stimuli. The user can be required to respond to audio stimuli within a predetermined time period in order to further test the cognitive performance of the user. For example, the user can be required to select a specific key on a keyboard or a hit a specific button on a controller in response to a buzzer. If the user selects the specified key/button within a predetermined period of time, points are added to the user's score. Otherwise, if the user selects the wrong key/button, or does not respond within the predetermined time period, points can be subtracted from the user's score.

In some implementations, the cognitive performance assessment game 102 can include both a training (variable difficulty) mode and a testing (static difficulty) mode. In the training mode, for example, the box spawn rate can increase as the user's score increases. This training mode can be used to tailor the cognitive performance assessment game 102 to the skill level of the user to better assist in detecting cognitive performance degradation of the user during testing or static difficulty mode. In the testing mode, for example, a set difficulty is chosen based on the results of one or more runs through the cognitive performance assessment game 102 by the user during the training mode. Keeping the difficulty of the cognitive performance assessment game 102 static during the testing mode can make it easier to detect changes in cognitive ability.

In some implementations, psychological metrics to be assessed can be modified with changes of in-game obstacles to meet the demands of an experiment. The cognitive performance assessment game 102 can display cognitive metrics of performance in real-time or near real-time. For example, tracked cognitive metric values can be updated on the display screen 100 about every 10^{th} of a second. These metrics can include but are not limited to: user score, accuracy, hand-eye coordination, speed, response time, spatial awareness, situational awareness, valuation, decision making, color detection proficiency and potentially auditory awareness. A replay of a session of the cognitive performance assessment game 102 can also be viewable afterwards and the performance can be analyzed in detail along with numerical data. In some implementations, the user score is calculated as a combination of one or more measured cognitive performance metrics. For example, speed, response time, and accuracy for the user can be tracked. These three metrics can then be used to calculate a single score metric for the user.

In some implementations, the cognitive performance assessment game 102 can be utilized to isolate external factors that can affect performance of a task.

In some implementations, the cognitive performance assessment game 102 can utilize an asymptotic learning curve. For example, to minimize the effect of learning, subjects can be given 10-20 minutes of training before the first scored tests. This can consist of a series of 3-9 runs on which a baseline performance assessment with the cognitive performance assessment game 102 can be established. During this training portion of the cognitive performance assessment game 102, the cognitive performance assessment game 102 can auto-scale to the user's skill level. For example, the cognitive performance assessment game 102 identifies the user's ability level and adjusts a difficulty level of future sessions of the cognitive performance assessment game 102 based on the identified ability level of the user. The game can utilize randomized targets to inherently minimize the effect of learning. For example, the boxes 104, 106, 110, 112, and 116 (and other displayed graphic objects) can be placed at random locations on the display screen 100. The training phase can be used to set a difficulty level of the cognitive performance assessment game 102 to the user's hand-eye coordination capabilities.

During testing runs of the cognitive performance assessment game 102, the cognitive performance assessment game 102 can be set to have a constant game difficulty level. For example, a maximum performance level of the user can be accurately estimated from the initial training performance runs performed by the user. A testing difficulty level can then be determined based on the identified maximum performance level. For example, during a testing run of the cognitive performance assessment game 102, game difficulty can be set to 75-85% of the user's max plateau performance level in order to better test performance stability, improvement, or degradation. In some implementations, difficulty does not change throughout the duration of the cognitive performance assessment game 102 for a particular user. This allows for a higher degree of certainty that any changes in score reflect actual changes in cognitive performance of the user rather (e.g., due to various environmental effects or impairment factors) than difficulty of the task changing. In some implementations, as a user becomes more familiar with the cognitive performance assessment game 102, through successive iterations of testing runs, the system can assess the user's improvement, and adjust the difficulty level for the user for future testing runs of the cognitive performance assessment game 102.

In some implementations, aspects of the cognitive performance assessment game 102 can be configured to diminish or eliminate mental fatigue of a user. For example, users can optimally focus on a specific task for short periods of time which can prevent the effects of mental fatigue on the user's performance. In some implementations, the effects of mental fatigue can be minimized by keeping each round of the cognitive performance assessment game 102 limited to a short time period, for example, 1 minute.

The competitive score system of the cognitive performance assessment game 102 can provide users with incentive to exceed previous performances as well as the performances of other users. For example, an online leader board listing scores for various users can be provided. In some implementations, the online leader board can be subdivided into different groups that have similar competitive interest. In some implementations, iterations of the cognitive performance assessment game 102 can be recorded for later viewing.

In some implementations, in addition to tracking user selections made by the user using a user input device, the system can also track eye movements/gaze direction of the user and display the user's gaze pattern as part of the cognitive performance assessment game 102. For example, the user can use a joystick to play the cognitive performance assessment game 102 by attempting to select boxes (e.g., boxes 104, 106, 110, 112, and 116) as they are displayed on the display screen 100. While the user is interacting with the cognitive performance assessment game 102 using the joystick, the user's eye motions can be tracked to determine the user's gaze direction. The cognitive performance assessment game 102 indicate on the display screen 100 the locations of the user's gaze as the user plays the cognitive performance assessment game 102. This information can be used to compare the user's gaze locations and selection locations to identify impairment or changes in cognitive performance for the user.

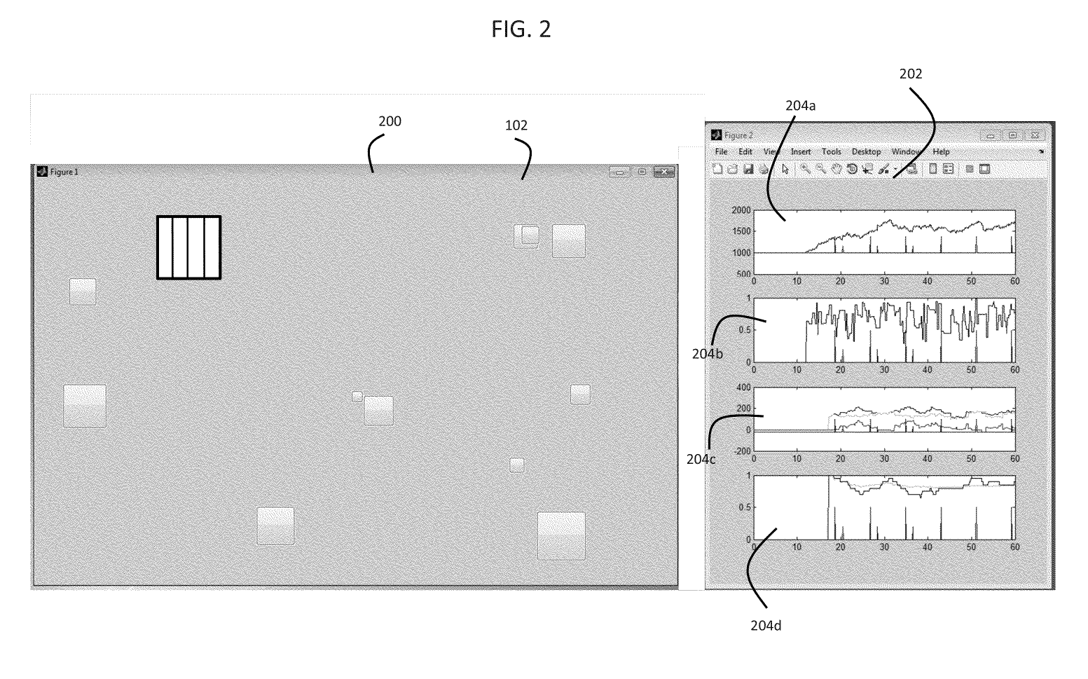

Turning to FIG. 2, a display screen 200 for the cognitive performance assessment game 102 is shown. As described above, the cognitive performance assessment game 102 includes several boxes that repeatedly spawn and must be targeted by the user. In some implementations, color differentiation skills of a user are tested during portions of the cognitive performance assessment game 102. The display screen 200 includes an analysis display section 202 that includes cognitive metric graphs 204a-d on the right side of the display. The cognitive metric graphs 204a-d can be used to display one or more metrics that are tracked by the cognitive performance assessment game 102. In some implementations, the cognitive metric graphs 204a-d are displayed as the cognitive performance assessment game 102 is being played and are updated in real-time as the user progresses through the game. In some implementations, the cognitive metric graphs 204a-d are rendered after the user has completed a round of the cognitive performance assessment game 102. Depending on the scenario and the aspect of cognitive performance evaluated, different metrics can be combined to get the best assessment of cognitive function.

Cognitive metrics to be reported in the analysis display section 202 that includes the cognitive metric graphs 204a-d can include user score, response time, accuracy, speed, total number of clicks by the user, total number of accurate user clicks, total number of inaccurate user clicks, total number of missed boxes, or overall performance. The analysis display section 202 can also display results of special heuristics including any of the above statistics separated into categories based on spawn distance between a newly spawned box and a cursor at the time the box is spawned, box color, or box disappearance rate (for scenarios in which boxes disappear at differing rates).

In the example shown, the cognitive metric graph 204a shows the user's score as the user progresses through the game. The score value fluctuates both up and down since the user can gain points for certain actions and lose points for other actions. In some implementations, a difficulty level of the cognitive performance assessment game 102 is selected such that a user will generally achieve a positive score at the end of a round of the cognitive performance assessment game 102 under normal conditions. In some implementations, the difficulty level is selected such that the user's final score is within a predetermined score range (e.g., between 1,500 and 2,000 points).

In the example shown, the cognitive metric graph 204b shows an accuracy assessment of the user's performance over the course of a round of the cognitive performance assessment game 102. The accuracy metric measures the ratio of user correct actions to incorrect actions (i.e. miss-clicks).

In the example shown, the cognitive metric graph 204c shows several tracked cognitive performance metrics displayed within the same graph. The cognitive metric graph 204c shows real-time tracking for total actions (e.g., selections or clicks) taken by the user displayed along with the number of incorrect actions (e.g., user missed a spawning target box) and correct actions (e.g., user successfully selected a spawning target box within the allotted time after spawning).

In the example shown, the cognitive metric graph 204d shows response time for a user over the course of a round of the cognitive performance assessment game 102. The response time metric measures the amount of time it takes users to target boxes from the instant they spawn. Response time can also be categorized into color based responses for attention and awareness focused tests. For example, response times for yellow boxes can be tracked separately from response times for grey and blue boxes.

Other tracked cognitive performance metrics can also be displayed as graphs or other graphic items within the analysis display section 202. For example, the analysis display section 202 can include a graph that shows overall performance of a user over the course of a round of the cognitive performance assessment game 102. The overall performance metric can be a continuously recorded score that combines values from the other metrics of performance and is capable of displaying a final output at the end of a round of the cognitive performance assessment game 102 in order to give the user or other observers a general idea of the user's cognitive performance during the game.

Other metrics that can be assessed can include speed. For example, the analysis display section 202 can include a graph showing the number of targets/actions that the user is able to perform over a fixed period of time.

The program also has the ability to interface with input physiologic metrics of the user to track total performance. Physiologic metrics that can be examined and tracked include heart rate, heart rate variability, pulse oxygen saturation, galvanic skin response, regional oxygen saturation (e.g. using a near-infrared spectroscopy (NIRS) device), eye motion/eye tracking, and motion of other portions of the body. In some implementations, other physiologic metrics of the user can be measured. For example, nervous ticks (foot tapping, excessive blinking, etc.) can be tracked by the program. In some implementations, one or more physiologic metrics can be combined with one or more cognitive performance metrics to produce the overall performance cognitive metric graph 204d.

Performance metrics and/or physiologic metrics measured by the cognitive performance assessment game 102 and/or a system implementing the cognitive performance assessment game 102 can be used to assess the effects of various environmental factors and other factors on the user's performance. For example, cognitive performance over a period of time can be assessed for users experiencing stress conditions including hypoxia (oxygen deprivation), hypothermia, sleep deprivation, mild to extreme fatigue, concussion, or other factors that may cause degradation in cognitive performance. The system implementing the cognitive performance assessment game 102 can also test the effects of various mental conditions or states on cognitive performance, including mild to extreme mental stress or pressure. Furthermore, the system can be used to identify when hypoxia has manifested in the cerebral circulation of an individual before they reach a critical phase of useful consciousness.

For example, the cognitive performance assessment game 102 can be provided to a user, and the user can engage in one or more testing iterations of the cognitive performance assessment game 102. During the testing iterations, one or more of the above described cognitive performance metrics can be tracked. In some implementations, physiologic metrics for the user can also be measured. The tracked cognitive performance metrics and/or physiologic metrics for the user can be stored as baseline metrics for the user under normal conditions (e.g., the user is not experiencing environmental or other stresses). The user can then be tested using the cognitive performance assessment game 102 at a later time (e.g., several hours, days, or weeks later). During the second testing period, the user can engage in one or more iterations of the cognitive performance assessment game 102 while experiencing one or more stress conditions.

For example, the user can be exposed to one or more environmental stresses either during or immediately preceding an iteration of the cognitive performance assessment game 102. Environmental stresses that can be applied to the user include lowering of the oxygen level of air being supplied to the user, lowering (or raising) atmospheric pressure of an environment in which the user is located, exposing the user to severe temperatures (e.g., extremely high temperatures, or extremely low, sub-freezing temperatures), or exposing the user to distracting stimuli external to the cognitive performance test, such as loud noises, bright and/or flashing lights, or unpleasant scents. In some implementations, other stresses on the user can be tested, such as sleep deprivation, hunger, concussion (testing after a user has experienced a concussion), or fatigue (for example, testing after the user has engaged in a strenuous or extenuated work out, or at a time that the user is experiencing workplace fatigue due to a long/stressful work day).

Cognitive performance metrics and/or physiologic metrics for the user can be tracked while the user is engaged in a session of the cognitive performance assessment game 102 while experiencing the one or more stress conditions. The tracked metrics for the user can be compared to the stored baseline metrics for the user to identify the effects of the one or more stress conditions on the user's performance. For example, a reduction in reaction time caused by sleep deprivation can be calculated. As another example, the effect of prolonged exposure to sub-freezing temperatures on the user's spatial awareness, accuracy, and hand-eye coordination can be identified.

In some implementations, information derived from comparing the metrics tracked for the user when experiencing stress conditions to baseline metric information for the user can be displayed to the user. In some implementations, the derived information can be displayed to other users (e.g., a test administrator). For example, the derived information can be displayed on a display screen other than the display screen used to display the cognitive performance assessment game 102 to the user being tracked. This can allow an administrator of the test to assess the effects of the one or more stress conditions on the user's performance. In some implementations, information regarding tracked metrics for the user that is displayed to the user differs from information regarding the tracked metrics for the user that is displayed to the test administrator.

The cognitive performance assessment game 102 can be administered to the user multiple times during multiple different testing sessions to track the effects of various different stress conditions on the user's performance. For example, after an initial testing session is conducted to track baseline cognitive performance information for the user, the user can engage in several iterations of the cognitive performance assessment game 102 during a second testing session while sleep deprived to identify the effects of sleep deprivation on the user. The user can later engage in another testing session using the cognitive performance assessment game 102 while being exposed to severe heat. The user can then engage in yet another testing session using the cognitive performance assessment game 102 while experiencing fatigue. The user can be successively tested in this manner while experiencing various different stress conditions or combinations of stress conditions to track the effects of each stress condition or combination of stress conditions on the user's cognitive performance.

In some implementations, the tracked cognitive performance metric information for the user can then be used to identify when the user is experiencing one or more stress conditions. For example, tracked cognitive performance metric information indicating the effects of low oxygen environments on the user can be used to identify when hypoxia has manifested in the cerebral circulation of the user prior to the user reaching a critical phase of useful consciousness. For example, the user can participate in a first set of one or more iterations of the cognitive performance assessment game 102 to identify baseline values for response time and accuracy for the user under normal conditions. The user can then participate in a second set of one or more iterations of the cognitive performance assessment game 102 while being exposed to a decreased oxygen level. The user's response time and accuracy can be measured while the user is experiencing decreased oxygen levels. Additionally, physiological measurements for the user can be taken, including tracking the user's blood oxygenation level using, for example, a pulse oximeter attached to the user's finger. The changes to the user's response time and accuracy, as measured by the system, can be compared to measured blood oxygenation level for the user to identify a correlation between the tracked response time and accuracy metrics for the user and changes in the user's blood oxygenation.

In some implementations, environmental factors or other stress conditions can be changed over the course of an iteration of the cognitive performance assessment game 102. For example, oxygen supplied to the user can be gradually decreased during a duration a particular instance of the cognitive performance assessment game 102. As another example, the oxygen supplied to the user can be decreased during a first time period of a particular iteration of the cognitive performance assessment game 102 and then increased (e.g., back to normal oxygen level, such as 21%) during a second time period of the particular iteration of the cognitive performance assessment game 102. As yet another example, environmental temperature can be gradually increased during a duration of a particular instance the cognitive performance assessment game 102. In some implementations, values for one or more environmental conditions can be displayed on the display screen 200 as part of the display of the cognitive performance assessment game 102. For example, if oxygen level is gradually changed during an iteration of the game, a graph can be displayed in the analysis display section 202 that indicates the changing oxygen levels over time. The changes in oxygen levels can then be easily compared to changes in tracked cognitive performance metrics for the user.

In some implementations, a user can engage in one or more iterations of the cognitive performance assessment game 102 while being exposed to stress conditions of varying severity. For example, the user can engage in successive iterations of the cognitive performance assessment game 102 while being exposed to gas mixtures having 8%, 9%, 10%, 12%, and 14% oxygen for successive different iterations. The cognitive performance assessment game 102 can also measure cognitive performance of users having been exposed to one or more stress conditions for varying degrees of time. For example, cognitive performance of the user can be measured by the cognitive performance assessment game 102 after the user has been exposed to decreased oxygen for two minutes and then again after the user has been exposed to decreased oxygen for six minutes. In some implementations, changes to stress conditions are automatically controlled by the system executing the cognitive performance assessment game 102. For example, the system can automatically execute multiple iterations of the cognitive performance assessment game 102 as the system also changes environmental temperature.

Later, cognitive performance metrics of the user (e.g., response time and accuracy) are measured, either during an iteration of the cognitive performance assessment game 102 or while the user is engaged in another task. Decreases in the user's response time and accuracy can indicate that the user is experiencing hypoxia. For example, the user's tracked cognitive performance metrics can be compared to previously tracked performance metric data for the user to identify when the user is experiencing the effects of hypoxia. In some cases, the tracked cognitive performance metric information can also be used to identify a time between detection of hypoxia for the user and the user reaching a critical phase of useful consciousness.

In some cases, reduced blood oxygenation for the user can be detected based on the tracked performance metrics prior to detection by a physiological metric measuring device, such as a pulse oximeter. For example, lag time between a user experiencing hypoxia and the detection of hypoxia by a pulse oximeter being worn by the user can be up to 15 to 30 seconds. This lag time is the amount of time it takes for lack of oxygen to manifest in the systemic circulation (where it can be detected by the pulse oximeter) as opposed to the time in which lack of oxygen manifests in the cerebral circulation. The cognitive performance assessment game 102 can detect degradation in the user's performance (and use that detected degradation to identify hypoxia in the user) prior to detection by a pulse oximeter.

In some implementations, tracked cognitive performance information for multiple users can be analyzed by the system to identify general trends in the effects of various stress conditions on the users. Additionally, the system can compare tracked cognitive performance information for multiple users to identify users that perform better than others when exposed to particular stress conditions. For example, the system can identify a first user as performing better than other users when suffering from sleep deprivation. The system can then identify a second user as performing better than other users when exposed to extreme cold. As another example, the system can identify users exhibiting the highest level of cognitive performance retention (compared to other users) when experiencing hypoxia. The results of this analysis can then be displayed for use by one or more users. For example, the information can be displayed to an administrator to aid the administrator in selecting one or more persons for engaging in a particular activity in which the participants may experience one or more stress conditions (e.g., a high altitude recovery mission).

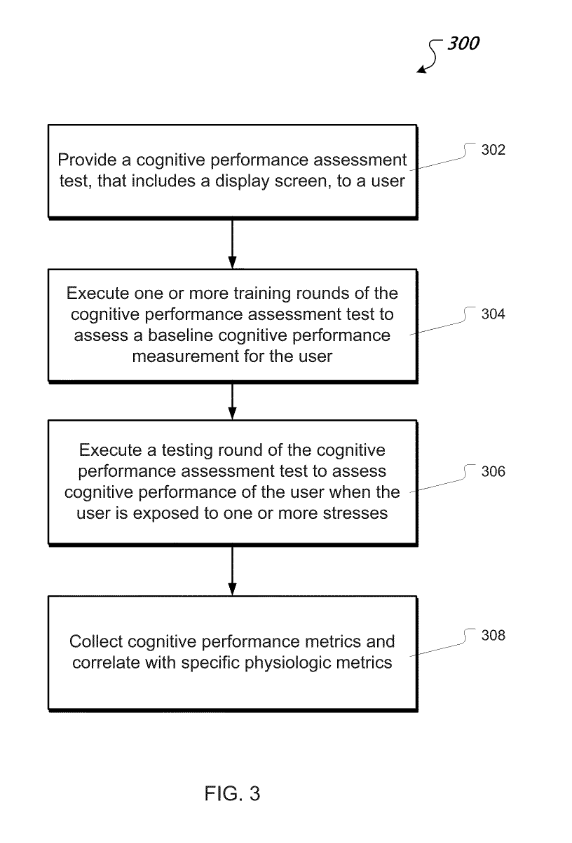

Turning now to FIG. 3, an example process 300 for using a cognitive performance assessment test (such as, e.g., the cognitive performance assessment game 102) to assess cognitive performance is shown. At stage 302, the process 300 provides a cognitive performance assessment test, that includes a display screen (such as, e.g., the display screen 200), to a user. For example, a user can utilize the cognitive performance assessment test using a personal computer, or a specialized testing system that includes a display screen.

At stage 304, the process 300 one or more training rounds of the cognitive performance assessment test to assess a baseline cognitive performance measurement for the user. For example, the user can run through three training rounds of the cognitive performance assessment test so that a baseline for cognitive performance can be determined for the user. In the training mode, for example, a difficultly level of the cognitive performance assessment test can increase as the user's score increases. For example, referring to the cognitive performance assessment game 102 described above, the box spawn rate can increase or the box disappearance time can decrease as the user's score increases. This training mode can be used to tailor the cognitive performance assessment test to the skill level of the user to better assist in detecting cognitive performance degradation of the user during a testing or static difficulty mode.

At stage 306, the process 300 executes a testing round of the cognitive performance assessment test to assess cognitive performance of the user when the user is exposed to one or more stresses. In the testing mode, for example, a set difficulty is chosen based on the results of one or more runs through the cognitive performance assessment test by the user during the training mode (stage 304). Keeping the difficulty of the cognitive performance assessment test static can make it easier to detect changes in cognitive ability. In some implementations, one or more stresses can be imparted on the user to test the user's cognitive performance while under stress. In some implementations, environmental stresses can be applied including, lowering of the oxygen level of air being supplied to the user, lowering (or raising) atmospheric pressure of an environment in which the user is located, exposing the user to severe temperatures (e.g., extremely high temperatures, or extremely low, sub-freezing temperatures), or exposing the user to distracting stimuli external to the cognitive performance assessment game 102, such as loud noises, bright and/or flashing lights, or unpleasant scents. In some implementations, other stresses on the user can be tested, such as sleep deprivation, hunger, or fatigue (for example, testing after the user has engaged in a strenuous or extenuated work out, or at a time that the user is experiencing workplace fatigue due to a long/stressful work day).

At stage 308, the process 300 collects cognitive performance metrics and correlates them with specific physiologic metrics to analyze results. Cognitive performance metrics can include user score, accuracy, hand-eye coordination, speed, response time, overall performance, spatial awareness, situational awareness, valuation, color recognition, decision making and potentially auditory awareness. In some implementations, one or more cognitive performance metrics are collected and displayed as the cognitive performance test is being played and are updated in real-time as the user progresses through the game. In some implementations, the cognitive performance metrics are determined after the user has completed a testing round of the cognitive performance assessment test. Physiologic metrics that can be tracked and compared to collected cognitive performance metrics can include heart rate, heart rate variability, pulse oxygen saturation, galvanic skin response, and regional oxygen saturation (NIRS).

Variations in various physiologic metrics can be compared to variations in cognitive performance to allow future identification of degradation of cognitive performance for a particular user based off of physiologic measurements. For example, changes in a user's heart rate and/or eye movements when the user is experiencing sleep deprivation (as compared to heart rate and/or eye movements during a baseline test for the user) can be tracked and compared to changes in the user's cognitive performance metrics during a testing round to identify correlations between the tracked cognitive performance metrics and the tracked physiologic metrics. After this comparison, tracked cognitive performance metrics for the user can be used to identify changes in physiologic metrics for the user, or vice versa. For example, changes in a tracked cognitive performance metric for the user during a testing round can be used to estimate changes in one or more physiologic metrics for the user.

Differences in various performance metrics can be identified to detect acute changes in cognitive function. For example, cognitive performance metrics collected during the testing round can be compared to baseline cognitive performance metrics identified during one or more training rounds of the cognitive performance assessment test. This comparison can identify differences in cognitive performance when the user is exposed to one or more stresses in comparison to the user's baseline performance. These differences can be used to detect acute changes in cognitive function and combine them with recorded changes in physiologic functions to give a more complete assessment of player condition. These identified differences for the user can be used in future scenarios to identify when the user is experiencing one or more stresses, such as, for example, hypoxia (or more particularly, hypoxia of the cerebral circulation system). Subsequent iterations of the testing rounds of the cognitive performance assessment test can be used to track the user's ability to improve functionality when exposed to one or more environmental or other stresses.

The features described in this disclosure can be implemented in digital electronic circuitry, or in computer hardware, firmware, software, or in combinations of them. The apparatus can be implemented in a computer program product tangibly embodied in an information carrier, e.g., in a machine-readable storage device, for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing context.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A computer-implemented method comprising:
providing, to a user and on a display screen of a computing system, a graphic user display interface for a cognitive performance assessment test;
displaying, on the display screen, a plurality of first game icons within the graphic user display interface at various time intervals, wherein each of said plurality of first game icons increases in size over the course of a display time period that does not exceed a first predetermined duration, and wherein each of said plurality of first game icons is removed from the graphic user display interface in response to one of i) being selected by the user or ii) the display time period reaching the first predetermined duration;
displaying, on the display screen, one or more second game icons on the graphic user display interface, the second game icons differing from the first game icons by at least one first display aspect, wherein each of the second game icons changes color if a second predetermined duration has elapsed after the second game icon is first displayed and prior to a user selection of the second game icon being received;
responsive to the user selecting a particular second game icon of the one or more second game icons prior to the second predetermined duration having elapsed after the particular second game icon is first displayed, displaying, on the display screen, a third game icon on the graphic user display interface, the third game icon differing from the first and second game icons by at least one second display aspect, wherein the third game icon disappears from the graphic user display interface after either i) being selected by the user or ii) a third predetermined duration from the third game icon being displayed has elapsed;
tracking values for at least one cognitive performance metric of the user during an iteration of the cognitive performance assessment test; and
displaying a visual indication of a change in values of the at least one cognitive performance metric over time.

2. The method of claim 1, wherein the at least one first display aspect is different from the at least one second display aspect.

3. The method of claim 1, wherein the at least one first display aspect is color.

4. The method of claim 1, further comprising:
receiving user input indicating a location within the graphic user display interface
determining that none of the plurality of first game icons, the one or more second game icons, or the third game icon is displayed at the location at the time that the user input is received;
in response to the determination, displaying a fourth game icon.

5. The method of claim 1, wherein the at least one cognitive performance metric comprises at least one of speed, response time, spatial awareness, and auditory awareness.

6. The method of claim 1, further comprising:
determining, using the tracked values for the at least one cognitive performance metric, that the user is experiencing decreased cognitive performance due to a stress condition experienced by the user;
storing, in a memory of the computing system, information indicating an association between the stress condition and decreased cognitive performance for the user; and
displaying information indicating a decrease in cognitive performance of the user due to the stress condition.

7. A computer-implemented method comprising:
providing, to a user and on a display screen of a computing system, a graphic user display interface for a cognitive performance assessment test;
executing, by the computing system, a first instance of the cognitive performance assessment test;
receiving, by the computing system, first user input from the user during the first instance of the cognitive performance assessment test;
determining a first set of one or more values for at least one cognitive performance metric using the received first user input;
executing, by the computing system, a second instance of the cognitive performance assessment test while the user is experiencing a stress condition;
receiving, by the computing system, second user input from the user during the second instance of the cognitive performance assessment test;
determining a second set of one or more values for the at least one cognitive performance metric using the received second user input;
comparing, by the computing system, the second set of values to the first set of values to determine an effect of the stress condition on cognitive performance of the user;
displaying an indication of the effect of the stress condition on cognitive performance of the user.

8. The method of claim 7, wherein executing the first instance of the cognitive performance assessment test comprises displaying, on the display screen, a plurality of game icons within the graphic user display interface, wherein each of said plurality of game icons increases in size over the course of a display time period that does not exceed a first predetermined duration, and wherein each of said plurality of game icons is removed from the graphic user display interface in response to one of i) being selected by the user or ii) the display time period reaching the first predetermined duration;
wherein receiving the first user input comprises receiving a selection of a particular game icon of the plurality of game icons prior to the display time period for the particular game icon reaching the first predetermined duration; and
wherein determining the first set of one or more values for the at least one cognitive performance metric comprises increasing a cognitive performance metric value in response to receiving the selection of the particular game icon prior to the display time period for the particular game icon reaching the first predetermined duration.

9. The method of claim 8, wherein receiving the first user input further comprises receiving an additional selection of a location within the graphic user display interface that does not include a game icon at the time that the additional selection is received; and
wherein determining the first set of one or more values for the at least one cognitive performance metric further comprises decreasing the cognitive performance metric value in response to receiving the additional selection.

10. The method of claim 7, wherein the stress condition is at least one of lowered oxygen level, altered air pressure, altered temperature, sleep deprivation, fatigue, and hunger.

11. The method of claim 7, wherein displaying the indication of the effect of the stress condition on cognitive performance of the user comprises displaying a numerical indication of the effect of the stress condition on cognitive performance of the user.

12. The method of claim 7, wherein displaying the indication of the effect of the stress condition on cognitive performance of the user comprises displaying a graphical indication of the effect of the stress condition on cognitive performance of the user.

13. The method of claim 7, further comprising:
executing, by the computing system, a third instance of the cognitive performance assessment test;
receiving, by the computing system, third user input from the user during the third instance of the cognitive performance assessment test;
determining a third set of one or more values for the at least one cognitive performance metric using the received third user input;
comparing, by the computing system, the third set of one or more values to the first and second sets of one or more values to identify a particular stress condition experienced by the user during the third instance of the cognitive performance assessment test;
displaying an indication of the particular stress condition experienced by the user during the third instance of the cognitive performance assessment test.

14. A computer storage medium encoded with a computer program, the program comprising instructions that when executed by one or more data processing apparatus cause the one or more data processing apparatus to perform operations comprising:
providing, to a user and on a display screen of a computing system, a graphic user display interface for a cognitive performance assessment test;
executing, by the computing system, a first instance of the cognitive performance assessment test;
receiving, by the computing system, first user input from the user during the first instance of the cognitive performance assessment test;
determining a first set of one or more values for at least one cognitive performance metric using the received first user input;
executing, by the computing system, a second instance of the cognitive performance assessment test while the user is experiencing a stress condition;
receiving, by the computing system, second user input from the user during the second instance of the cognitive performance assessment test;
determining a second set of one or more values for the at least one cognitive performance metric using the received second user input;
comparing, by the computing system, the second set of values to the first set of values to determine an effect of the stress condition on cognitive performance of the user;
displaying an indication of the effect of the stress condition on cognitive performance of the user.

15. The computer storage medium of claim 14, wherein executing the first instance of the cognitive performance assessment test comprises displaying, on the display screen, a plurality of game icons within the graphic user display interface, wherein each of said plurality of game icons increases in size over the course of a display time period that does not exceed a first predetermined duration, and wherein each of said plurality of game icons is removed from the graphic user display interface in response to one of i) being selected by the user or ii) the display time period reaching the first predetermined duration;
wherein receiving the first user input comprises receiving a selection of a particular game icon of the plurality of game icons prior to the display time period for the particular game icon reaching the first predetermined duration; and
wherein determining the first set of one or more values for the at least one cognitive performance metric comprises increasing a cognitive performance metric value in response to receiving the selection of the particular game icon prior to the display time period for the particular game icon reaching the first predetermined duration.

16. The computer storage medium of claim 15, wherein receiving the first user input further comprises receiving an additional selection of a location within the graphic user display interface that does not include a game icon at the time that the additional selection is received; and
wherein determining the first set of one or more values for the at least one cognitive performance metric further comprises decreasing the cognitive performance metric value in response to receiving the additional selection.

17. The computer storage medium of claim 14, wherein the stress condition is at least one of lowered oxygen level, altered air pressure, altered temperature, sleep deprivation, fatigue, and hunger.

18. The computer storage medium of claim 14, wherein displaying the indication of the effect of the stress condition on cognitive performance of the user comprises displaying a numerical indication of the effect of the stress condition on cognitive performance of the user.

19. The computer storage medium of claim 14, wherein displaying the indication of the effect of the stress condition on cognitive performance of the user comprises displaying a graphical indication of the effect of the stress condition on cognitive performance of the user.

20. The computer storage medium of claim 14, the operations further comprising:
executing, by the computing system, a third instance of the cognitive performance assessment test;
receiving, by the computing system, third user input from the user during the third instance of the cognitive performance assessment test;
determining a third set of one or more values for the at least one cognitive performance metric using the received third user input;
comparing, by the computing system, the third set of one or more values to the first and second sets of one or more values to identify a particular stress condition experienced by the user during the third instance of the cognitive performance assessment test;
displaying an indication of the particular stress condition experienced by the user during the third instance of the cognitive performance assessment test.
